(19) United States
(12) Patent Application Publication
(10) Pub. No.: US 2026/0324987 A1
(43) Pub. Date: Oct. 22, 2026

(54) NEONATAL CONTINUOUS CARDIAC OUTPUT MONITORING

(71) Applicant: Draeger Medical Systems, Inc., Andover, MA (US)

(72) Inventor: Mark Starlin Vaughn, Honca Path, SC (US)

(21) Appl. No.: 19/157,643

(22) PCT Filed: Feb. 23, 2024
(86) PCT No.: PCT/IB2024/051776

§ 371 Date: Aug. 18, 2025

Related U.S. Application Data
(60) Provisional application No. 63/448,119, filed on Feb. 24, 2023.

(51) Int. Cl. A61B 5/02; A61B 5/00; A61B 5/0205

(52) U.S. Cl. CPC A61B 5/02028; A61B 5/02055; A61B 5/6852; A61B 5/6871; A61B 5/7435; A61B 2503/04

(57) ABSTRACT

A method for neonatal monitoring includes: catheterizing a neonatal patient through an implanted umbilicus interface; injecting a signaling fluid into the neonatal patient through the neonatal PiCCO venous catheter; monitoring the circulating blood for a hemodynamic signal through the neonatal PiCCO arterial catheter; and detecting the hemodynamic signal. Catheterizing the neonatal patient through an implanted umbilicus interface, includes: inserting a neonatal PiCCO arterial catheter into an umbilical artery of the neonatal patient; and inserting a neonatal PiCCO venous catheter into an umbilical vein of the neonatal patient.

### CROSS REFERENCE TO RELATED APPLICATIONS

The present application claims priority to and the benefit of U.S. Prov. Pat. App. Ser. No. 63/448,119, which was filed on Feb. 24, 2023, for all purposes, including the right of priority, which application is hereby incorporated herein by reference in its entirety and to the extent that is not inconsistent with the present disclosure.

### TECHNICAL FIELD

The present disclosure pertains to hemodynamic monitoring and, more particularly, neonatal hemodynamic monitoring.

### DESCRIPTION OF THE RELATED ART

This section of this document introduces information about and/or from the art that may provide context for or be related to the subject matter described herein and/or claimed below. It provides background information to facilitate a better understanding of the various aspects of the present invention. This is a discussion of “related” art. That such art is related in no way implies that it is also “prior” art. The related art may or may not be prior art. The discussion in this section of this document is to be read in this light, and not as admissions of prior art.

One aspect of patient care derives from a field known as “hemodynamics”. Hemodynamics is a term used to describe how the blood flows through the arteries and veins of the body and the forces that affect the blood flow. Hemodynamic factors, or factors affecting blood flow, for an individual patient can include factors such as blood vessel diameter, friction from the blood vessel walls and deposits thereon, blood vessel expansion and contraction, cardiac output, heart rate, cardiac ventricular efficacy, among others. Note that this list is representative and is neither necessarily exclusive. nor necessarily exhaustive. Each of these factors may exhibit a high degree of variability based on a patient's unique physiology.

The art includes a number of techniques for hemodynamic monitoring of patients. Hemodynamic monitoring has proven useful in a clinical setting for a number of reasons including the fact that it can sometimes be used in monitoring and/or diagnosing complex medical conditions. Hemodynamics are known to be related to a number of wide-ranging conditions such as atherosclerosis, hypertension, hypotension, heart valve issues, heart failure, peripheral artery disease, deep vein thrombosis, and cardiac arrest. Again, this list is neither exclusive nor exhaustive. Accordingly, knowledge of a patient's hemodynamics may provide insight to one or more of these conditions and perhaps others as well.

Hemodynamic monitoring techniques are generally classed as “invasive” or “non-invasive”. Invasive techniques may include measuring various arterial or venous blood pressures using catheters. Cardiac catheterization may be used to measure the pressure on either side of a cardiac valve for heart valve issues, for example. Non-invasive techniques may include echocardiograms, electrocardiograms, and/or blood pressure cuffs. Some monitoring techniques may employ both invasive and non-invasive techniques. For instance, cardiac output may be measured in some instances using both a pulmonary artery catheter and a transthoracic echocardiogram.

One hemodynamic technique is called “Pulse Contour Cardiac Output” monitoring, or “Pulse Index Continuous Cardiac Output” monitoring(“PiCCO”). PiCCO combines a transpulmonary thermodilution technique with a pulse contour analysis. The former calculates volumetric measurements of preload and cardiac output while the latter provides continuous cardiac output and stroke volume variation. PiCCO includes inserting a central venous pressure catheter and an arterial thermodilution line. The arterial thermodilution line may generally be placed in any one of a number of arteries including the axillary, brachial, femoral, or radial artery. Generally, a radial insertion is used, which calls for a longer catheter. The PiCCO arterial catheter is used to sense various hemodynamic parameters quantifying various hemodynamic factors, which can then be analyzed to arrive at still other hemodynamic parameters.

Unfortunately, the situation for neonatal patients differs. A survey of the difficulties in neonatal hemodynamic monitoring is set forth in Fister, Petja, Grosek, Štefan, “Hemodynamic Monitoring in Neonates”. Selected Topics in Neonatal Care, edited by R. Barría, IntechOpen, 2017. 10.5772/intechopen.69215. As set forth therein, neonatal patients are often hemodynamically unstable and such instability is a significant concern, often leading the neonatal intensive care unit (“NICU”) admission. Although some useful data from known techniques, most of them have not been shown to be effective for neonatal hemodynamic monitoring.

### SUMMARY

In a first aspect, a method for neonatal monitoring comprises: catheterizing a neonatal patient through an implanted umbilicus interface; injecting a signaling fluid into the neonatal patient through the neonatal PiCCO venous catheter; monitoring the circulating blood for a hemodynamic signal through the neonatal PiCCO arterial catheter; and detecting the hemodynamic signal. Catheterizing the neonatal patient through an implanted umbilicus interface, includes: inserting a neonatal Pulse Contour Cardiac Output (“PiCCO”) arterial catheter into an umbilical artery of the neonatal patient; and inserting a neonatal PiCCO venous catheter into an umbilical vein of the neonatal patient.

In a second aspect, a neonatal hemodynamic monitoring apparatus comprises: a data input; a user interface; and a hemodynamic monitoring controller. The hemodynamic monitoring controller includes: a processor-based resource; and a memory encoded with instructions. The instructions, when executed by the processor-based resource, perform a method comprising: receiving a hemodynamic signal from a neonatal Pulse Contour Cardiac Output (“PiCCO”) arterial catheter inserted into an artery of a neonatal patient, the hemodynamic signal being responsive to the injection of a signaling fluid through a neonatal PiCCO venous catheter inserted into the vein of a neonatal patient, the insertion of the PiCCO arterial catheter and the PiCCO venous catheter occurring through an implanted umbilicus interface; determining a plurality of neonatal patient hemodynamic parameters from the received hemodynamic signal; determining one or more hemodynamic conditions indicated by the neonatal patient hemodynamic parameters; and displaying one or more of the determined hemodynamic parameters, or one or more hemodynamic conditions indicated by the one or more hemodynamic factors, or a combination thereof.

In a third aspect, a computer-implemented method for hemodynamic monitoring comprises: receiving a hemodynamic signal from a neonatal Pulse Contour Cardiac Output (“PiCCO”) arterial catheter inserted into an artery of a neonatal patient, the hemodynamic signal being responsive to the injection of a signaling fluid through a neonatal PiCCO venous catheter inserted into the vein of a neonatal patient, the insertion of the PiCCO arterial catheter and the PiCCO venous catheter occurring through an implanted umbilicus interface; determining a plurality of neonatal patient hemodynamic parameters from the received hemodynamic signal; determining one or more hemodynamic conditions indicated by the neonatal patient hemodynamic parameters; and displaying one or more of the determined hemodynamic parameters, or one or more hemodynamic conditions indicated by the one or more hemodynamic factors, or a combination thereof.

In a fourth aspect, a neonatal hemodynamic monitoring kit comprises: a signaling fluid dispenser; a neonatal catheterization kit; and an umbilicus interface for use in catheterizing a neonatal patient using the neonatal catheterization kit. The neonatal catheterization kit includes: a neonatal Pulse Contour Cardiac Output (“PiCCO”) arterial catheter by which a hemodynamic signal may be detected; and a neonatal PiCCO venous catheter through which a signaling fluid may be injected.

In a fifth aspect, a method for neonatal monitoring is substantially as shown and described.

In a sixth aspect, a method for neonatal hemodynamic monitoring is substantially as shown and described.

In a seventh aspect, a computer-implemented method for hemodynamic monitoring is substantially as shown and described.

In an eighth aspect, a neonatal hemodynamic monitoring kit is substantially as shown and described.

In a ninth aspect, a neonatal hemodynamic monitoring apparatus is substantially as shown and described.

The above presents a simplified summary of the invention in order to provide a basic understanding of some aspects of the invention. This summary is not an exhaustive overview of the invention. It is not intended to identify key or critical elements of the invention or to delineate the scope of the invention. Its sole purpose is to present some concepts in a simplified form as a prelude to the more detailed description that is discussed later.

### BRIEF DESCRIPTION OF THE DRAWINGS

The invention may be understood by reference to the following description taken in conjunction with the accompanying drawings, in which like reference numerals identify like elements, and in which:

FIG. 1 conceptually illustrates the blood circulatory system of a representative neonatal patient having an implanted umbilicus interface.

FIG. 2 conceptually depicts a neonatal hemodynamic monitoring kit in accordance with one or more embodiments.

FIG. 3 is a schematic block diagram an assembled neonatal monitoring apparatus in accordance with one or more embodiments.

FIG. 4 schematically represents a neonatal monitoring apparatus deployed to hemodynamically monitor a neonatal patient in accordance with one or more embodiments.

FIG. 5 depicts a neonatal monitoring apparatus in accordance with one or more embodiments.

While the invention is susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION

Illustrative embodiments of the subject matter claimed below will now be disclosed. In the interest of clarity, not all features of an actual implementation are described in this specification. It will be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

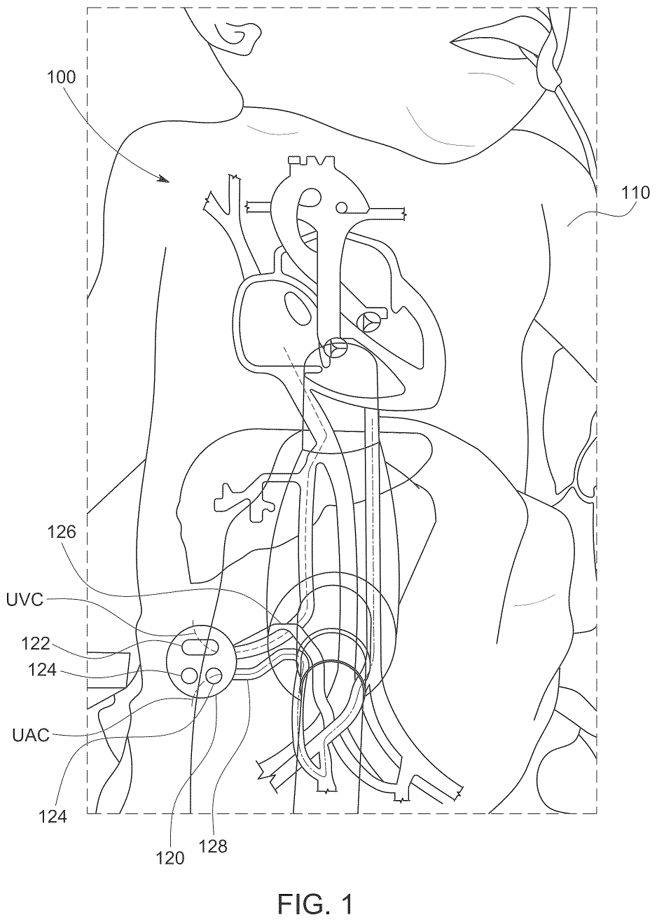

FIG. 1 conceptually illustrates the blood circulatory system 100 of a representative neonatal patient 110 having an implanted umbilicus interface 120. Superimposed on the depiction of the neonatal patient 110 is a conceptualized diagram of internal organs of the neonatal patient 110, including veins and arteries. Although it is anticipated that the presently disclosed apparatus and method will be used to hemodynamically monitor unwell neonatal patients, this will not be the case in all embodiments.

The umbilicus interface 120 includes a plurality of ports including at least an umbilicus venous catheter (“UVC”) port 122 and two umbilicus arterial catheter (“UAC”) ports 124. The umbilical interface 120 is implanted in the umbilicus (not otherwise shown) of the neonatal patient to provide hygienic catheterization access to the umbilical vein 126 and the umbilical artery 128.

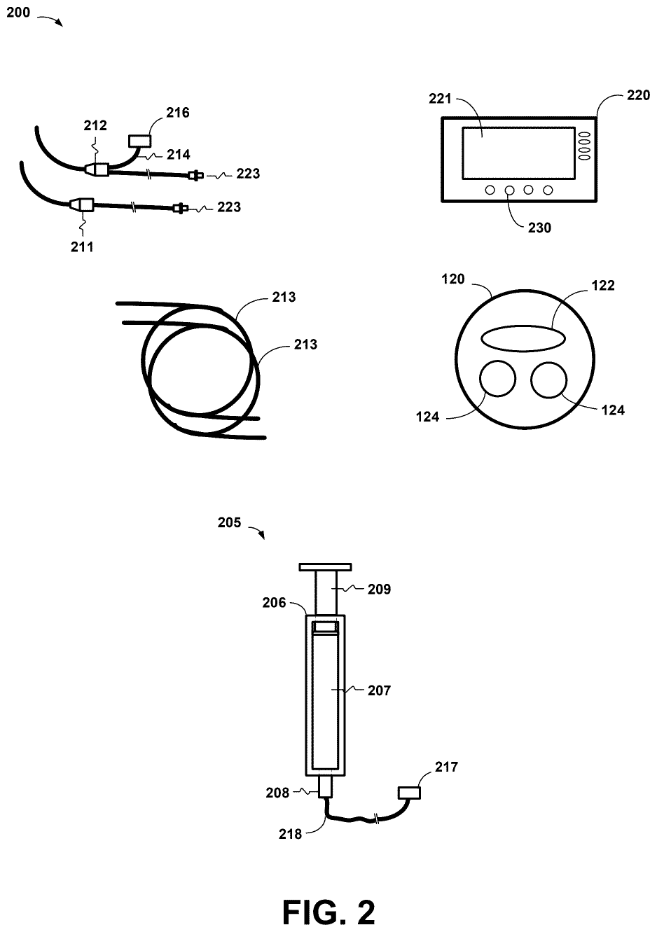

FIG. 2 conceptually depicts a neonatal hemodynamic monitoring kit 200 in accordance with one or more embodiments. The neonatal hemodynamic monitoring kit 200 includes, in this particular embodiment, a signaling fluid dispenser 205, a neonatal catheterization kit 210, an umbilicus interface 120 as discussed above, and a neonatal hemodynamic monitor 220. One or more of the signaling fluid dispenser 205, an umbilicus interface 120, and a neonatal hemodynamic monitor 220 may be omitted in some embodiments. Kit 200 may optionally include suitable packaging effective for sterilization and storage and transport of the contents. Additional contents may be additionally included in alternative embodiments without departing from the scope and spirit of the invention.

The signaling fluid dispenser 205 is an injection-type dispenser including a body 206 defining an injectate well 207 and terminating in a tip 208. The signaling fluid dispenser 205 also includes a plunger 209. The plunger 209 sealably engages the wall of the injectate well 207 as it is depressed to expel a signaling fluid (not shown) from the signaling fluid dispenser 205 through the tip 208.

Note that the signaling fluid dispenser 205 disclosed herein is, by way example and illustration, but one means for dispensing the signaling fluid as described below. Other embodiments may use equivalent structures that perform the function of dispensing the signaling fluid. For example, a signaling fluid dispenser may include more than one injectate well in some embodiments.

The neonatal catheterization kit 210 includes a neonatal PiCCO arterial catheter 211 and a neonatal PiCCO venous catheter 212. In use, as will be discussed further below, the PiCCO arterial catheter is used to detect a hemodynamic signal when inserted into the neonatal patient. The neonatal PiCCO venous catheter 211 through which a signaling fluid may be injected is similarly used to inject the signaling fluid into the neonatal patient's blood circulatory system.

A neonatal PiCCO catheter, whether arterial or venous, is a catheter that may be used in PiCCO monitoring but is sized for neonatal veins and arteries and designed to be inserted through an implanted umbilicus interface. To some degree, size of the catheter will depend on the size of the arteries and veins of the neonatal patient. The patient's weight may be used as a proxy for this consideration. For example, at least one guideline recommends umbilical arterial catheters of 5 Fr for infants over 1500 grams, 3.5 Fr for infants less than 1500 grams inclusive, and 2.5 Fr for infants who cannot accommodate 3.5 Fr.

As shown in FIG. 2, both the neonatal PiCCO arterial catheter 211 and the neonatal PiCCO venous catheter 212 include an electrical lead 213 terminating in an electrical connector 223 over which information may be exchanged with the neonatal hemodynamic monitor 220 when assembled therewith. The electrical leads 213 may be implemented in smart PiCCO cables. In some embodiments, the neonatal PiCCO arterial catheter 211 and the neonatal PiCCO venous catheter 212 may exchange information with the neonatal hemodynamic monitor 220 wirelessly. In such embodiments, the neonatal PiCCO arterial catheter 211 and the neonatal PiCCO venous catheter 212 may include wireless transceivers (not shown) or transmitters (not shown) and the neonatal hemodynamic monitor 220 may also include wireless transceivers (not shown) or a wireless receiver (not shown).

Although not shown, both the neonatal PiCCO arterial catheter 211 and the neonatal PiCCO venous catheter 212 also include adaptors to interface with existing PiCCO smart cables. The term “smart cable” as used herein means that the cables include elements that are programmed and/or programmable.

The neonatal PiCCO arterial catheter 211 also includes a fluid lead 214 terminating in a fluid connector 216. The fluid connector 216 mates with the fluid connector 217 of the fluid delivery line 218 through which the signaling fluid dispenser 205 delivers the signaling fluid to the neonatal PiCCO arterial catheter 211 when assembled and in use.

The neonatal hemodynamic monitor 220 may be a general purpose medical monitor modified to perform the functionalities disclosed herein or may be purpose-built specifically therefor. The medical monitor 220 will generally have a display 221 of some sort and some means for electrically or electromagnetically communicating with the PiCCO arterial catheter 211 and a neonatal PiCCO venous catheter 212. Those means might be, for example, one or more ports 230 to provide an electrical connection over the leads 213 of the neonatal PiCCO arterial catheter 211 and the neonatal PiCCO venous catheter 212.

Note that the neonatal hemodynamic monitor 220 disclosed herein is, by way of example and illustration, but one means for receiving, processing, and displaying information as described below. Other embodiments may use equivalent structures that perform the function of receiving, processing, and displaying information. For example, the neonatal hemodynamic monitor 220 disclosed herein incorporates the display 221. However, in other embodiments, the display may be separated from the rest of the neonatal hemodynamic monitor and information may be exchanged over a wired or wireless connection.

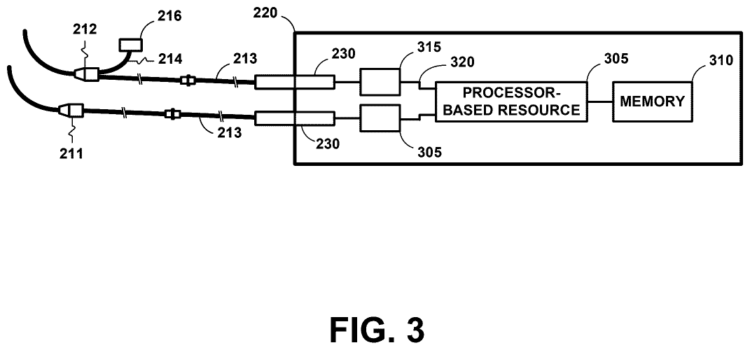

FIG. 3 is a schematic block diagram an assembled neonatal monitoring apparatus 300 in accordance with one or more embodiments. The assembled neonatal monitoring apparatus 300 includes the signaling fluid dispenser 205, the neonatal PiCCO arterial catheter 211, the neonatal PiCCO venous catheter 212, and the neonatal hemodynamic monitor 220. Some embodiments may include the umbilicus interface 120 in addition to those components shown in FIG. 3. The illustrated embodiment, however, presumes that the umbilicus interface 120 is already installed and awaiting assembly into the neonatal monitoring apparatus 300 by catheterization of the neonatal patient.

The neonatal PiCCO arterial catheter 211, the neonatal PiCCO venous catheter 212 are shown assembled with the neonatal hemodynamic monitor 220 through a wired electrical connection formed by mating the connectors 223 to the ports 230. The neonatal PiCCO arterial catheter 211 and the signaling fluid dispenser 205 through the mating of the fluid connectors 216, 217. Alternative embodiments (not shown) may include other components that may be assembled to various parts of the assembled neonatal monitoring apparatus 300. For example, some embodiments may have temperature sensors (not shown) or blood pressure sensors (not shown) that may be electrically connected to the neonatal hemodynamic monitor 220. Those in the art having the benefit of this disclosure may appreciate still other variations of this kind.

FIG. 3 also conceptually illustrates selected aspects of the neonatal hemodynamic monitor 220. The neonatal hemodynamic monitor 220 is an electronic device and includes a processor-based resource 305 executing instructions stored in a memory 310. The memory 310 may be on-chip memory on the processor-based resource 305, a separate memory device, or a combination thereof. The neonatal hemodynamic monitor 220 also includes conditioning circuitry 315 for the data inputs and/or command outputs and, as mentioned above, the display 221. The neonatal hemodynamic monitor 220 further includes a bus system 320 over which the various electronic components communicate with one another.

The processor-based resource 305 may comprise one or more hardware processors, where each hardware processor may have a single or multiple processor cores. The processor-based resource may therefore be a processor, such as a microprocessor, a controller, a microcontroller, a central processing unit (“CPU”), application specific integrated circuits (“ASICs”), field-programmable gate arrays (“FPGAs”), digital signal processors (“DSPs”), etc. as are known in the art. Or, the processor-based resource 305 may be a processor chipset containing multiple processors comprising one or more of the processor types listed immediately above, perhaps also including one or more of a graphics processing unit (“GPU”) and/or a math co-processor.

The processor-based resource 305 may include at least one shared cache that stores data (e.g., computing instructions) that are utilized by one or more other components of processor-based resource 305 and may comprise a portion of the memory 310. For example, the shared cache may be a locally cached data stored in a memory for faster access by components of the processing elements that make up processor-based resource 305. In one or more embodiments, the shared cache may include one or more mid-level caches, such as level 2 (L2), level 3 (L3), level 4 (L4), or other levels of cache, a last level cache (LLC), or combinations thereof.

The memory 310 may also include off-chip memory operatively and communicatively coupled to processor 305 over the bus system 320. Memory 310 may be a non-transitory medium configured to store various types of data. For example, memory 310 may include one or more storage devices 1320 that comprise a non-volatile storage device and/or volatile memory. Volatile memory, such as random-access memory (“RAM”), can be any suitable non-permanent storage device. The non-volatile storage devices 1320 can include one or more disk drives, optical drives, solid-state drives (“SSDs”), tap drives, flash memory, read only memory (“ROM”), and/or any other type of memory designed to maintain data for a duration of time after a power loss or shut down operation. In certain instances, the non-volatile storage devices (not separately shown) may be used to store overflow data if allocated RAM is not large enough to hold all working data. The non-volatile storage devices may also be used to store programs that are loaded into the RAM when such programs are selected for execution.

Persons of ordinary skill in the art are aware that software programs may be developed, encoded, and compiled in a variety of computing languages for a variety of software platforms and/or operating systems and subsequently loaded and executed by processor-based resource 305. In one embodiment, the compiling process of the software program may transform program code written in a programming language to another computer language such that the processor-based resource 305 is able to execute the programming code. For example, the compiling process of the software program may generate an executable program that provides encoded instructions (e.g., machine code instructions) for processor-based resource 305 to accomplish specific, non-generic, particular computing functions.

After the compiling process, the encoded instructions may then be loaded as computer executable instructions or process steps to processor-based resource 305 from memory 310. Processor-based resource 305 may be configured to execute the stored instructions or process steps in order to perform instructions or process steps to transform the computing device into a non-generic, particular, specially programmed machine or apparatus. Stored data, e.g., may be accessed by processor-based resource 305 during the execution of computer executable instructions or process steps.

Still referring to FIG. 3, the display 221, the display can be implemented in various ways, including by a liquid crystal display (“LCD”) or a cathode-ray tube (“CRT”) or light emitting diode (“LED”) display, such as an organic light emitting diode (“OLED”) technologies. The display 221 may include a touchscreen (not shown) which may be a part of a user interface. Information may be displayed as text, graphics, or a combination thereof.

Persons of ordinary skill in the art are aware that the computing device 1300 may comprise other components well known in the art, such as sensors, powers sources, and/or analog-to-digital converters, not explicitly shown in FIG. 3. For example, the neonatal hemodynamic monitor 220 may include a user interface. A user interface can include not only the display 221, but also one or more of a positional input device (such as a mouse, touchpad, touchscreen, or the like), a keyboard, buttons, dials, or other forms of user input and output devices, none of which are shown. The user interface components may also be communicatively coupled to processor-based resource 305.

The neonatal hemodynamic monitor 220 is also programmed/configured with a variety of software. Among the software stored in memory 310 is a PiCCO module 325 and a hemodynamic analysis (“HA”) module 330. The term “module” is used in its accustomed meaning in the art to describe hardware, or software, or a combination thereof assigned or dedicated to a particular functionality. In the illustrated embodiment, the PiCCO module 325 and the HA module 330 are software executed by the processor-based processor 305 to perform the associated functionalities as described below. As discussed above, other software may and typically will be stored in the memory 310 but are not shown. Such additional software may include, but are not limited to, user interface software, data conditioning software, an operating system, etc.

The memory 310 may also be encoded with and a store of data 335 that may be associated with the procedure described herein or in the PiCCO and/or hemodynamic analyses. Data may be manually entered, electronically collected, or pulled from electronic medical records. The data store 335 may include one or more data structures containing the data and may be selected to best suit the data. Examples of data structures may include, but are not limited to, databases, linked lists, lookup tables, or a tree.

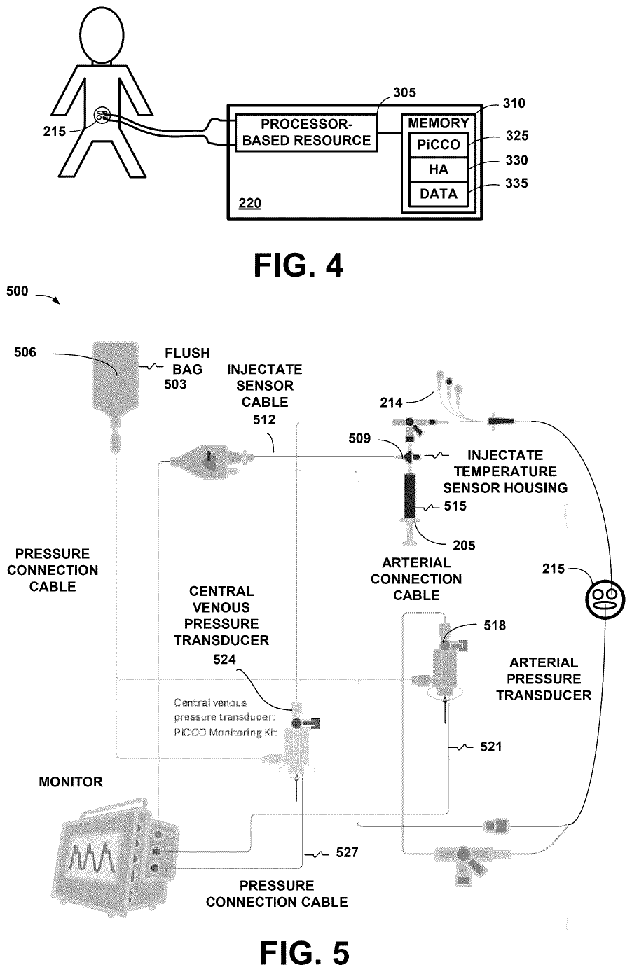

FIG. 4 schematically represents a neonatal monitoring apparatus 300 deployed to hemodynamically monitor a neonatal patient 303 in accordance with one or more embodiments. The umbilical interface 120 is shown implanted in the neonatal patient 110. The umbilical interface 120 may be pre-installed or installed as a part of the technique described herein. The implantation of the umbilical interface 120 may be performed in any suitable manner known to the art.

The weight of the neonatal patient 110 is known or is taken. For example, the neonatal patient 303 may be positioned on a scale that outputs a reading on the weight of the neonatal patient 110 to the neonatal hemodynamic monitor 220 or a wired or wireless connection. For another example, the neonatal patient 110 may be removed from the neonatal monitoring apparatus 300 to a remote location (e.g., another room), weighed, and then the weight manually entered into the neonatal hemodynamic monitor 220 through the user interface. For yet another example, the neonatal patient 110 may have been recently weighed and the neonatal hemodynamic monitor 220 may pull the weight from an electronic medical record for the neonatal patient 110. Still other embodiments may utilize some combination of parts of these approaches.

The weight of the neonatal patient 110 may have many uses in the disclosed technique. As mentioned above, the weight may be used to size the neonatal PiCCO arterial catheter 211 and the neonatal PiCCO venous catheter 212 to be used in the procedure. Some embodiments may also use the weight as a proxy to determine whether the neonatal patient 110 is an infant, a juvenile, or an adult. This determination may also consider other physiological parameters such as sex and height. This determination may, in turn, then inform the values to be used in various PiCCO and other hemodynamic analyses. Whether the patient is an infant, juvenile, or adult may also be manually entered or pulled from an electronic medical record.

There are a number of treatment and patient care considerations that may be addressed by actions taken that are not discussed herein. These actions are not discussed because they are not germane to the technique currently disclosed, for clarity, and so as not to obscure that which is claimed below. For example, it may be desirable to restrain the neonatal patient 110. Or, precautions may be taken to prevent hypothermia in the neonatal patient 110, such as laying the neonatal patient 110 on an exothermic mattress or the use of a radiant warmer. Yet another example is that it may be desirable to flush the neonatal PiCCO arterial catheter 211, the neonatal PiCCO venous catheter 212, and/or the signaling fluid dispenser 205 with a sterile saline solution. Those in the art having the benefit of this disclosure will be able to recognize the desirability of and perform these and other tasks ancillary to the disclosed technique.

Similarly, there may aspects of the PiCCO monitoring process that are not discussed for the same or similar reasons. For example, may PiCCO monitoring processes include blood pressure monitoring. There may also be calibration practices or display options, or many other options for the PiCCO monitoring process depending upon the particular embodiment. These actions are not discussed because they are not germane to the technique currently disclosed, for clarity, and so as not to obscure that which is claimed below.

A medical practitioner (not shown)—whether a nurse's assistant, a nurse, a physician's assistant, or a physician, for instance—then catheterizes the neonatal patient 303 with the neonatal PiCCO arterial catheter 211, the neonatal PiCCO venous catheter 212. Those in the art having the benefit of this disclosure will appreciate that ordinarily there are two umbilical arteries whereas there is only a single neonatal PiCCO arterial catheter 211. The unused umbilical artery may be clamped or otherwise terminated during the procedure disclosed herein.

Once the neonatal patient 110 is catheterized, a signaling fluid is injected from the signaling fluid dispenser 205 into the patient through the neonatal PiCCO arterial catheter 211. The signaling fluid may be, for example, a sterile saline solution in the amount of 15 mL cooled to a predetermined temperature. The predetermined temperature is less than the nominal core temperature of the neonatal patient 110.

It is generally preferred to finish the injection in a relatively short period, such as seven seconds, as shorter injection times tend to yield better signals. The time at which the signaling fluid is injected is recorded by the neonatal hemodynamic monitor 220. The neonatal PiCCO arterial catheter 211 may include a micro-temperature probe (not shown) or sensor (not shown) that can sense the predetermined temperature of the signaling fluid. This procedure may be repeated several times. In one embodiment, the process is repeated three times.

The micro-temperature probe or sensor of the neonatal PiCCO arterial catheter 211 may continually sense temperature of the blood in the umbilical artery and may transmit the sensed temperature to the neonatal hemodynamic monitor 220 over the respective PiCCO smart cable 400a. When the signaling fluid is injected into the umbilical artery at the predetermined temperature, the sensed temperature of the blood in the umbilical artery will dip, or temporarily decrease, as the signaling fluid cools the blood. The neonatal hemodynamic monitor 220, through the PiCCO module 325 (shown in FIG. 3), may begin a timer upon recognizing the sensed temperature as the start of the injection. Alternatively, the time of injection may be manually entered into the neonatal hemodynamic monitor 220.

The blood cooled by the signaling fluid transits the arterial system of the neonatal patient 110, eventually reaching the neonatal PiCCO venous catheter 212. The neonatal PiCCO venous catheter 212 may also include a micro-temperature probe (not shown) or sensor (not shown). The neonatal PiCCO venous catheter 212 continually senses the temperature of the blood in the umbilical vein as the blood circulates through the arterial system of the neonatal patient 110.

The sensed temperature of the blood in the umbilical vein will dip, or temporarily decrease, as the signaling fluid earlier cooled the blood. Those in the art having the benefit of this disclosure will appreciate that cooled blood will warm as it transits the arterial system. Thus, the predetermined temperature of the signaling fluid should be sufficiently less than the nominal core temperature of the neonatal patient 110 to produce a distinguishably cooler temperature at the neonatal PiCCO venous catheter 212. In one embodiment, the signaling fluid is at least 10 °C below blood temperature and no warmer than 24 °C. The signaling fluid therefore may be refrigerated or cooled on ice prior to use.

The temperature sensed by the neonatal PiCCO venous catheter 212 may be transmitted to the neonatal hemodynamic monitor 220 over the respective PiCCO smart cable 400b. The drop in the sensed temperature at the neonatal PiCCO venous catheter 212 signals to the PiCCO module 325 (shown in FIG. 3) that the transit is complete. The PiCCO module 325 then determines the transit time of the cooled blood. The PiCCO module 325 then performs a PiCCO analysis using the determined transit time and other information such as the weight of the neonatal patient 110.

The PiCCO analysis generally includes at least two parts. The first is arterial pulse contour analysis and the second is transpulmonary thermodilution. The arterial pulse contour analysis provides continuous, dynamic information. The transpulmonary thermodilution provides static measurement used to calibrate the continuous pulse contour parameters. Parameters that may be considered includes, for example and without limitation, continuous cardiac output and stroke volume. Saline fluid drip (as a first step) or blood pressure medicine (as a second step) could be used to improve stroke volume, for instance.

For example, in the PiCCO analysis some embodiments may determine PiCCO parameters including one or more of pulse contour parameters, transpulmonary thermodilution parameters, and physiological parameters. Pulse contour parameters may include, for instance, pulse contour cardiac output, continuous cardiac index, stroke volume, stroke volume index, systemic vascular resistance, systemic vascular resistance index, index of left ventricular contractility, stroke volume variation, and pulse pressure variation. Example transpulmonary thermodilution parameters may include cardiac output, cardiac index, global end diastolic volume, global end diastolic volume index, extravascular lung water, extravascular lung water index, global ejection fraction, pulmonary vascular permeability index, cardiac function index, intrathoracic blood volume, and intrathoracic blood volume index. Physiological parameters may include body surface area, predicted body weight, and predicted body surface area.

The physiological parameters are calculated body characteristics that are used to index various other PiCCO volumetric parameters. The calculations for some of these physiological parameters depend, in part, on whether the patient is an adult, a juvenile, or an infant. (Factors such as height, weight, and sex are also sometimes used.) Accordingly, many of the PiCCO parameters depend either directly or indirectly on the patient's status in this respect.

One particular embodiment therefore automatically determines whether the patient is an adult, juvenile, or infant. This automatic determination may be performed by the neonatal hemodynamic monitor 220. The automatic determination may be made from the physiological parameters, for example, by some calculation using a weighted formula or a lookup table indexed by the physiological parameters. Or, the information may be pulled from a medical record stored in some data structure, such as a database, stored on the neonatal hemodynamic monitor 220 or accessed through some distributed computing environment.

The illustrated embodiment also performs additional hemodynamic monitoring with the PiCCO monitoring described above. As shown in FIG. 3, The neonatal hemodynamic monitor 220 includes a hemodynamic analysis module 330 for this purpose. Various embodiments may monitor one or more of oxygen delivery, oxygen consumption, central venous oxygen saturation, cardiac output, arterial oxygen content, stroke volume, heart rate, oxygenation, hemoglobin, preload, afterload, contractility, and pulmonary edema.

FIG. 5 schematically represents a neonatal monitoring apparatus 500 deployed to hemodynamically monitor a neonatal patient in accordance with one or more embodiments. The neonatal monitoring apparatus 500 shares common parts with earlier disclosure and like parts bear like numbers.

However, the neonatal monitoring apparatus also includes a flush bag 503 containing a flush fluid 506 for flushing the various components after assembly and prior to use. Also, an injectate temperature sensor housing 509 senses the temperature of the signaling fluid 515 as it is injected and communicates with the neonatal hemodynamic monitor 220 over the injectate sensor cable 512. An arterial pressure transducer 518 senses the arterial pressure and communicates the sensed pressure to the neonatal hemodynamic monitor 220 over the cable 521. Finally, a central venous pressure transducer 524 senses pressure and transmits it to the neonatal hemodynamic monitor 220 over the pressure connection cable 527.

Thus, in a first embodiment, a method for neonatal monitoring comprises: catheterizing a neonatal patient through an implanted umbilicus interface, injecting a signaling fluid into the neonatal patient through a neonatal PiCCO venous catheter; monitoring the circulating blood for a hemodynamic signal through a neonatal PiCCO arterial catheter; and detecting the hemodynamic signal. Catheterizing the neonatal patient includes inserting the neonatal PiCCO arterial catheter into an umbilical artery of the neonatal patient; and inserting the neonatal PiCCO venous catheter into an umbilical vein of the neonatal patient.

In a second embodiment, the method of the first embodiment further comprises implanting the umbilicus interface in the neonatal patient.

In a third embodiment, the method of the first embodiment further comprises monitoring for hemodynamic parameters.

In a fourth embodiment, injecting the signaling fluid in the first embodiment includes injecting a predetermined amount of a signaling saline solution at a predetermined temperature.

In a fifth embodiment, the hemodynamic signal in the fourth embodiment is a temperature fluctuation in the circulating blood.

In a sixth embodiment, the method of the first embodiment further comprises: determining a plurality of neonatal patient hemodynamic parameters from the received hemodynamic signal; determining one or more hemodynamic conditions indicated by the neonatal patient hemodynamic parameters; and displaying one or more of the determined hemodynamic parameters, or one or more hemodynamic conditions indicated by the one or more hemodynamic factors, or a combination thereof.

In a seventh embodiment, the method of the first embodiment further comprises implanting the umbilicus interface in the neonatal patient.

In an eighth embodiment, the method of the first embodiment further comprises: determining a plurality of neonatal patient hemodynamic parameters from the detected hemodynamic signal; determining one or more hemodynamic conditions indicated by the neonatal patient hemodynamic parameters; and displaying one or more of the determined hemodynamic parameters, or one or more hemodynamic conditions indicated by the one or more hemodynamic factors, or a combination thereof.

In a ninth embodiment, a neonatal hemodynamic monitoring apparatus comprises: a data input; a user interface; and a hemodynamic monitoring controller. The hemodynamic monitoring controller includes a processor-based resource and a memory. The memory is encoded with instructions that, when executed by the processor-based resource, perform a method comprising: receiving a hemodynamic signal from a neonatal PiCCO arterial catheter inserted into an artery of a neonatal patient, the hemodynamic signal being responsive to the injection of a signaling fluid through a neonatal PiCCO venous catheter inserted into the vein of a neonatal patient, the insertion of the PiCCO arterial catheter and the PiCCO venous catheter occurring through an implanted umbilicus interface; determining a plurality of neonatal patient hemodynamic parameters from the received hemodynamic signal; determining one or more hemodynamic conditions indicated by the neonatal patient hemodynamic parameters; and displaying one or more of the determined hemodynamic parameters, or one or more hemodynamic conditions indicated by the one or more hemodynamic factors, or a combination thereof.

In a tenth embodiment, the neonatal hemodynamic monitoring apparatus of the ninth embodiment further comprises monitoring for hemodynamic parameters.

In an eleventh embodiment, injecting the signaling fluid in the ninth embodiment includes injecting a predetermined amount of a signaling saline solution at a predetermined temperature.

In a twelfth embodiment, the hemodynamic signal in the ninth embodiment is a temperature fluctuation in the circulating blood.

In a thirteenth embodiment, the hemodynamic conditions in the ninth embodiment include monitoring for hemodynamic parameters.

In a fourteenth embodiment, a computer-implemented method for hemodynamic monitoring comprises: receiving a hemodynamic signal from a neonatal PiCCO arterial catheter inserted into an artery of a neonatal patient, the hemodynamic signal being responsive to the injection of a signaling fluid through a neonatal PiCCO venous catheter inserted into the vein of a neonatal patient, the insertion of the PiCCO arterial catheter and the PiCCO venous catheter occurring through an implanted umbilicus interface; determining a plurality of neonatal patient hemodynamic parameters from the received hemodynamic signal; determining one or more hemodynamic conditions indicated by the neonatal patient hemodynamic parameters; and displaying one or more of the determined hemodynamic parameters, or one or more hemodynamic conditions indicated by the one or more hemodynamic factors, or a combination thereof.

In a fifteenth embodiment, the computer-implemented method of the fourteenth embodiment further comprises monitoring for hemodynamic parameters.

In a sixteenth embodiment injecting the signaling fluid in the fourteenth embodiment includes injecting a predetermined amount of a signaling saline solution at a predetermined temperature.

In an seventeenth embodiment, the hemodynamic signal of the fourteenth embodiment is a temperature fluctuation in the circulating blood.

In a eighteenth embodiment, the hemodynamic conditions in the fourteenth embodiment include monitoring for hemodynamic parameters.

In a nineteenth embodiment, a neonatal hemodynamic monitoring kit, comprises: a signaling fluid dispenser; a neonatal catheterization kit; and an umbilicus interface for use in catheterizing a neonatal patient. The neonatal cauterization kit includes a neonatal PiCCO arterial catheter by which a hemodynamic signal may be detected; and a neonatal PiCCO venous catheter through which a signaling fluid may be injected; and In a twentieth embodiment, the neonatal hemodynamic monitoring kit of the nineteenth embodiment further comprises a hemodynamic monitor that, in use: receives a hemodynamic signal from the neonatal PiCCO arterial catheter; and performs a hemodynamic analysis on the hemodynamic signal.

In a twenty-first embodiment, the hemodynamic monitor of the twentieth embodiment further comprises: a processor-based resource; and a memory encoded with instructions that, when executed by the process, performs the hemodynamic analysis.

In a twenty-second embodiment, in the neonatal hemodynamic monitoring kit of the twenty-first embodiment, the hemodynamic monitor further comprises a user interface. Also, the memory is encoded instructions such that the method performed when executed by the processor-based resource further includes: receiving user commands through the user interface; and displaying one or more hemodynamic parameters or one or more patient conditions indicated by the one or more hemodynamic parameters, or a combination thereof.

In a twenty-third embodiment, the neonatal hemodynamic monitoring kit of the twenty-second embodiment further comprises: a data input; and a user interface through the displaying is performed.

A twenty-fourth embodiment is a method for neonatal monitoring substantially as shown and described herein.

A twenty-fifth embodiment is a method for neonatal hemodynamic monitoring substantially as shown and described herein.

A twenty-sixth embodiment is a computer-implemented method for hemodynamic monitoring substantially as shown and described herein.

A twenty-seventh embodiment is a neonatal hemodynamic monitoring kit substantially as shown and described herein.

A twenty-eighth embodiment is a neonatal hemodynamic monitoring apparatus substantially as shown and described herein.

This concludes the detailed description. The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

1. A method for neonatal monitoring, comprising:
catheterizing a neonatal patient through an implanted umbilicus interface, including:
inserting a neonatal Pulse Contour Cardiac Output (“PiCCO”) arterial catheter into an umbilical artery of the neonatal patient; and
inserting a neonatal PiCCO venous catheter into an umbilical vein of the neonatal patient;
injecting a signaling fluid into the neonatal patient through the neonatal PiCCO venous catheter;
monitoring the circulating blood for a hemodynamic signal through the neonatal PiCCO arterial catheter; and
detecting the hemodynamic signal.

2. The method of claim 1, further comprising implanting the umbilicus interface in the neonatal patient.

3. The method of claim 1, further comprising monitoring for hemodynamic parameters.

4. The method of claim 1, wherein injecting the signaling fluid includes injecting a predetermined amount of a signaling saline solution at a predetermined temperature.

5. The method of claim 4, wherein the hemodynamic signal is a temperature fluctuation in the circulating blood.

6. The method of claim 1, further comprising:
determining a plurality of neonatal patient hemodynamic parameters from the received hemodynamic signal;
determining one or more hemodynamic conditions indicated by the neonatal patient hemodynamic parameters; and
displaying one or more of the determined hemodynamic parameters, or one or more hemodynamic conditions indicated by the one or more hemodynamic factors, or a combination thereof.

7. The method of claim 1, further comprising implanting the umbilicus interface in the neonatal patient.

8. The method of claim 1, further comprising:
determining a plurality of neonatal patient hemodynamic parameters from the detected hemodynamic signal;
determining one or more hemodynamic conditions indicated by the neonatal patient hemodynamic parameters; and
displaying one or more of the determined hemodynamic parameters, or one or more hemodynamic conditions indicated by the one or more hemodynamic factors, or a combination thereof.

9. A neonatal hemodynamic monitoring apparatus, comprising:
a data input;
a user interface; and
a hemodynamic monitoring controller, including:
a processor-based resource; and
a memory encoded with instructions that, when executed by the processor-based resource, performs a method comprising:
receiving a hemodynamic signal from a neonatal Pulse Contour Cardiac Output (“PiCCO”) arterial catheter inserted into an artery of a neonatal patient, the hemodynamic signal being responsive to the injection of a signaling fluid through a neonatal PiCCO venous catheter inserted into the vein of a neonatal patient, the insertion of the PiCCO arterial catheter and the PiCCO venous catheter occurring through an implanted umbilicus interface;
determining a plurality of neonatal patient hemodynamic parameters from the received hemodynamic signal;
determining one or more hemodynamic conditions indicated by the neonatal patient hemodynamic parameters; and
displaying one or more of the determined hemodynamic parameters, or one or more hemodynamic conditions indicated by the one or more hemodynamic factors, or a combination thereof.

10. The neonatal hemodynamic monitoring apparatus of claim 9, further comprising monitoring for hemodynamic parameters.

11. The neonatal hemodynamic monitoring apparatus of claim 9, wherein injecting the signaling fluid includes injecting a predetermined amount of a signaling saline solution at a predetermined temperature.

12. The neonatal hemodynamic monitoring apparatus of claim 9, wherein the hemodynamic signal is a temperature fluctuation in the circulating blood.

13. The neonatal hemodynamic monitoring apparatus of claim 9, wherein the hemodynamic conditions include monitoring for hemodynamic parameters.

14-18. (canceled)

19. A neonatal hemodynamic monitoring kit, comprising:
a signaling fluid dispenser;
a neonatal catheterization kit, including:
a neonatal Pulse Contour Cardiac Output (“PiCCO”) arterial catheter by which a hemodynamic signal may be detected; and
a neonatal PiCCO venous catheter through which a signaling fluid may be injected; and
an umbilicus interface for use in catheterizing a neonatal patient.

20. The neonatal hemodynamic monitoring kit of claim 19, further comprising a hemodynamic monitor that, in use:
receives a hemodynamic signal from the neonatal PiCCO arterial catheter; and
performs a hemodynamic analysis on the hemodynamic signal.

21. The neonatal hemodynamic monitoring kit of claim 20, wherein the hemodynamic monitor further comprises:
a processor-based resource; and
a memory encoded with instructions that, when executed by the process, performs the hemodynamic analysis.

22. The neonatal hemodynamic monitoring kit of claim 21, wherein:
the hemodynamic monitor further comprises a user interface; and
the memory is encoded instructions such that the method performed when executed by the processor-based resource further includes:
receiving user commands through the user interface; and
displaying one or more hemodynamic parameters or one or more patient conditions indicated by the one or more hemodynamic parameters, or a combination thereof.

23. The neonatal hemodynamic monitoring kit of claim 22, further comprising:
a data input; and
a user interface through the displaying is performed.

24-28. (canceled)

29. The neonatal hemodynamic monitoring kit of claim 19, further comprising a neonatal hemodynamic monitoring apparatus, comprising:
a data input;
a user interface; and
a hemodynamic monitoring controller, including:
a processor-based resource; and
a memory encoded with instructions that, when executed by the processor-based resource, performs a method comprising:
receiving a hemodynamic signal from a neonatal Pulse Contour Cardiac Output (“PiCCO”) arterial catheter inserted into an artery of a neonatal patient, the hemodynamic signal being responsive to the injection of a signaling fluid through a neonatal PiCCO venous catheter inserted into the vein of a neonatal patient, the insertion of the PiCCO arterial catheter and the PiCCO venous catheter occurring through an implanted umbilicus interface;
determining a plurality of neonatal patient hemodynamic parameters from the received hemodynamic signal;
determining one or more hemodynamic conditions indicated by the neonatal patient hemodynamic parameters; and
displaying one or more of the determined hemodynamic parameters, or one or more hemodynamic conditions indicated by the one or more hemodynamic factors, or a combination thereof.

30. The neonatal hemodynamic monitoring kit of claim 29, wherein the hemodynamic signal is a temperature fluctuation in the circulating blood.
